(19) United States
(12) Plant Patent Application Publication
(10) Pub. No.: US 2015/0181788 P1
(43) Pub. Date: Jun. 25, 2015

(54) Magnolia plant named 'JURMAG5'

(50) ;

(71) Applicant: Mark Jury, Waitara (NZ)

(72) Inventor: Mark Jury, Waitara (NZ)

(21) Appl. No.: 13/998,945

(22) Filed: Dec. 20, 2013

(51) Int. Cl. A01H 5/00

(52) U.S. Cl. PLT/216

(57) ABSTRACT

A new and distinct Magnolia cultivar named ‘JURMAG5’ is disclosed, characterized by having a compact habit, slower growth and large orange-yellow flowers with distinctly rounded petals. The new variety is a Magnolia, normally produced as an ornamental plant.

### LATIN NAME OF THE GENUS AND SPECIES

Magnolia hybrid

### VARIETY DENOMINATION

‘JURMAG5’

### BACKGROUND OF THE INVENTION

The new cultivar is a product of a planned breeding program. The new variety resulted from a crossing performed in 1993 in North Taranaki, Nth Is, New Zealand. The seed parent is the unpatented variety, Magnolia accuminala ‘Yellow Bird’. The pollen parent is the unpatented variety Magnolia hybrid lolanthe'. The new variety was selected 2002, by the inventor, Mark Jury at Mark Jury Nursery, 591 Otaroa Road Waitara, North Taranaki, Nth Is, New Zealand in a group of seedlings resulting from the 1993 crossing.

Asexual reproduction of the new cultivar ‘JURMAG5’ by vegetative cuttings was first performed at a commercial nursery in New Zealand during 2002 and has shown that the unique features of this cultivar are stable and reproduced true to type on successive generations.

### SUMMARY OF THE INVENTION

The cultivar ‘JURMAG5’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘JURMAG5’ These characteristics in combination distinguish ‘JURMAG5’ as a new and distinct Magnolia cultivar:
1. Compact growth habit.
2. Creamy yellow colored petals
3. Growth habit less vigorous than other yellow flowering Magnolias
4. Distinctive rounded petals.
5. Large flower size

### PARENT COMPARISON

Plants of the new cultivar ‘JURMAG5’ are similar to plants of the seed parent, Magnolia ‘Yellow’ in most horticultural characteristics, however, plants of the new cultivar ‘JURMAG5’ have significantly larger flowers than the seed parent variety.

Plants of the new cultivar ‘JURMAG5’ are similar to plants of the pollen parent, Magnolia ‘Iolanthe’ in most horticultural characteristics, however, plants of the new cultivar ‘JURMAG5’ have creamy yellow flowers, whereas the pollen parent variety has pink and white flowers.

### BRIEF DESCRIPTION OF THE PHOTOGRAPHS

The accompanying photograph in FIG. 1 illustrates in full color a typical young flowers of ‘JURMAG5” outdoors.

FIG. 2 illustrates in full color typical mature flowers of ‘JURMAG5’. Age of the plant producing the flowers is approximately 20 years. The photographs were taken using conventional techniques and although colors may appear different from actual colors due to light reflectance it is as accurate as possible by conventional photographic techniques.

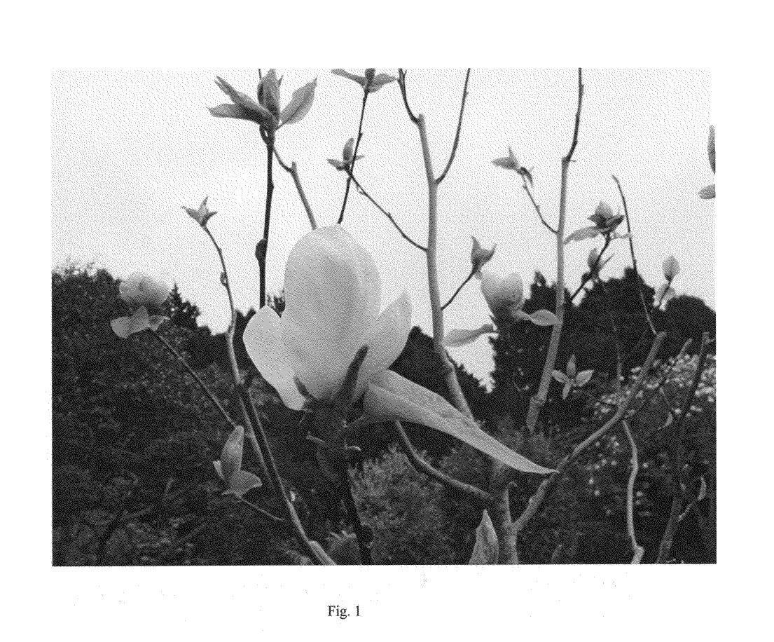

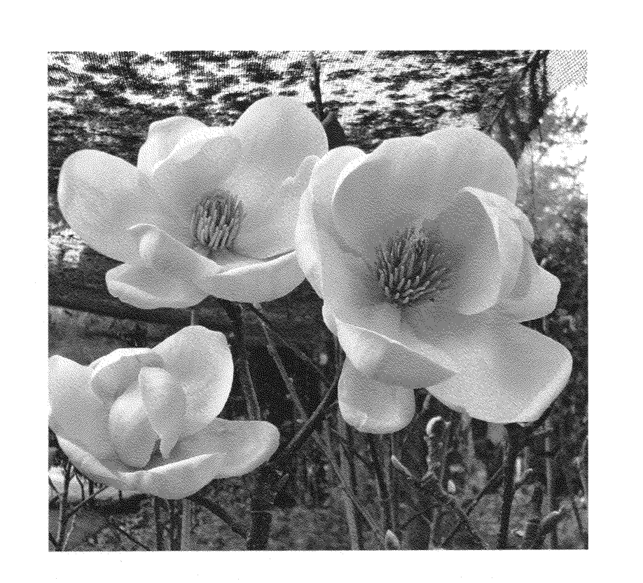

### DETAILED BOTANICAL DESCRIPTION

In the following description, color references are made to The Royal Horticultural Society Colour Chart 2007 except where general terms of ordinary dictionary significance are used. The following observations and measurements describe ‘JURMAG5’ plants grown outdoors in New Zealand. Growing conditions are similar to USDA zone 7. Measurements and numerical values represent averages of typical plant types.
Botanical classification: Magnolia hybrid ‘JURMAG5.’
Propagation:
Time to initiate roots: About 60 days at approximately 21° C.
Time to produce a rooted cutting: About120 days at 21° C.
Root description: Typical Magnolia root, thick, coarse, and white.
Plant:
Growth habit: Compact, upright tree.
Age of plant described: 20 years.
In a container or in the ground: Ground.
Height: Approximately 4 meters.
Plant spread: Approximately 3 meters.
Growth rate: Slow to average
Length of primary lateral branches: Approximately 50 cm.
Diameter of lateral branches: Aproximately 15 cm.
Quantity of lateral branches: Approximately 20
Branching arrangement: Sub-opposite.
Trunk:
   Diameter.—Approximately 18 cm, after 20 years.
   Texture.—Smooth.
   Color.—Near RHS Greyed-Green 189C.
Leaf:
   Arrangement.—Alternate.
   Quantity.—Approximately 8 per main branch.
   Average length.—Approximately 20 cm.
   Average width.—Approximately 13 cm.
   Shape of blade.—Ovate.
   Apex.—Acuminate.
   Base.—Cuneate.
   Margin.—Entire.
   Texture of top surface.—Smooth.
   Texture of bottom surface.—Moderately pubescent.
   Pubescence.—Moderate, present only on underside.
   Leaf internode length.—Approximately 3 cm.
   Color.—Young, foliage upper side: Near RHS Green 139D Young foliage under side: Near RHS Green 139D Mature foliage tipper side: Near RHS Green 137A. Mature foliage under side: Near RHS Green 143A.
   Venation.—Type: Pinnate Venation color upper side: Near RHS Green 137B Venation color under side: Near RHS Green 143B.
   Petiole.—Average Length: Approximately 1.5 cm. Color: Near RHS Green 141 B Diameter: Approximately 3 cm.
Flower:
Natural flowering season: August through September, in the Southern hemisphere.
Begins flowering after how many years/months: 1 year from bud stage
Inflorescence type and habit: Singly occurring, overlapping cupping, flowers.
Rate of flower opening: Approximately 6 days from bud to fully opened flower.
Flower longevity on plant: 4 to 5 days, after fully opened.
Quantity of flowers: Approximately 50 open flowers per tree
Flower size:
   Diameter.—Approximately 12 cm.
   Height.—Approximately 8 cm.
Pedicel:
   Length.—1 cm.
   Diameter.—0.8 cm.
   Color.—Near RHS Yellow-Green 144C.
Petals:
   Petal arrangement.—whorled, overlapping.
   Size.—Length: Range from approximately 5.0 cm to 8.0 cm. Width: Range from approximately 5.0 cm to 8.0 cm.
   Margin.—Entire
   Apex.—Rounded
   Petal quantity.—12.
   Texture.—Smooth.
Color:
   Petals.—When opening: Upper surface: Near RHS Yellow-Orange 18D Lower surface: Near RHS Yellow-Orange 18D Fully opened: Upper surface: Near RHS Yellow-Orange 18C Lower surface: Near RHS Yellow-Orange 18C.
   Bud.—Shape: Ovate Length: Approximately 5 cm. Diameter: Approximately 2 cm. Color: Near RHS Yellow 11 D.
   Sepals/tepals.—Shape: Lanceolate. Quantity: 12 Length: Approximately 1 cm. Width: Approximately 0.5 cm. Margin: Entire. Texture: Smooth.
Reproductive organs:
Stamens:
   Number.—Approximately 35.
   Filament length.—Approximately 1.5 cm.
   Filament color.—Near RHS Yellow 10A.
Anthers:
   Length.—Approximately 0.7 cm.
   Shape.—Linear.
   Color.—Colored by moderately produced pollen, Yellow-range 20A.
Pistil:
   Number.—1.
   Length.—Approximately 1.5 cm.
   Style.—Length: Approximately 1.4 cm. Color: Near RHS Yellow 2C.
Other characteristics:
Seeds and fruits: The new cultivar does not set seed. Female sterile.
Disease/pest resistance: Neither resistance nor susceptibility to pathogens and pests common to Magnolia have been observed.
Temperature tolerance: The new variety is suitable for growing in USDA zones 3 through 8.

What is claimed is:

1. A new and distinct cultivar of Magnolia plant named ‘JURMAG5’ as herein illustrated and described.
